(19) United States
(12) Patent Application Publication
(10) Pub. No.: US 2014/0114227 A1
(43) Pub. Date: Apr. 24, 2014

(54) LYMPHEDEMA MEDICAL DEVICE

(71) Applicant: Boston Scientific Scimed, Inc., Maple Grove, MN (US)

(72) Inventors: Nathan Zamarripa, Sudbury, MA (US); Jeff Gray, Sudbury, MA (US)

(73) Assignee: Boston Scientific Scimed, Inc., Maple Grove, MN (US)

(21) Appl. No.: 14/054,322

(22) Filed: Oct. 15, 2013

Related U.S. Application Data
(60) Provisional application No. 61/716,168, filed on Oct. 19, 2012.

(51) Int. Cl. A61M 27/00

(52) U.S. Cl. CPC A61M 27/002
USPC 604/8

(57) ABSTRACT

Medical devices and methods for making and using medical devices are disclosed. An example medical device may include a medical device for moving lymphatic fluid. The medical device may include an implantable body having an inlet end region and an outlet end region. The inlet end region may include one or more inlet opening. The outlet end region may include one or more outlet openings. A pump member may be positioned between the inlet end region and the outlet end region. The pump member may be configured to draw lymphatic fluid into the implantable body through the inlet end region and transfer lymphatic fluid out from the implantable body through the outlet end region.

### CROSS-REFERENCE TO RELATED APPLICATIONS

This application claims priority under 35 U.S.C. §119 to U.S. Provisional Application Ser. No. 61/716,168, filed Oct. 19, 2012, the entirety of which is incorporated herein by reference.

### TECHNICAL FIELD

The present disclosure pertains to medical devices, and methods for manufacturing medical devices. More particularly, the present disclosure pertains to medical devices for use along the lymphatic system.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. An example medical device may include a medical device for moving lymphatic fluid. The medical device may include an implantable body having an inlet end region and an outlet end region. The inlet end region may include one or more inlet opening. The outlet end region may include one or more outlet openings. A pump member may be positioned between the inlet end region and the outlet end region. The pump member may be configured to draw lymphatic fluid into the implantable body through the inlet end region and transfer lymphatic fluid out from the implantable body through the outlet end region.

Another example medical device may include a lymphedema medical device. The lymphedema medical device may include an implantable tubular member having one or more fluid openings formed therein. The implantable tubular member may be configured to be implanted within a lymphatic vessel region of a patient. The lymphedema medical device may also include pump coupled to the implantable tubular member. The pump may be configured to draw lymphatic fluid into the implantable tubular member through the one or more fluid openings. An outlet tube may be coupled to the pump.

A method for moving lymphatic fluid is also disclosed. The method may include providing a lymphedema medical device. The lymphedema medical device may include an implantable tubular member having one or more fluid openings formed therein, a pump coupled to the implantable tubular member, the pump being configured to draw lymphatic fluid into the implantable tubular member through the one or more fluid openings, and an outlet tube coupled to the pump. The method may also include implanting the implantable tubular member within a lymphatic vessel region of a patient and activating the pump. Activating the pump may draw lymphatic fluid into the implantable tubular member through the one of more fluid openings and to the outlet tube.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a partially cross-sectional side view of an example medical device;

FIG. 2 is a partially cross-sectional side view of the example medical device illustrated in FIG. 1 disposed in a body lumen;

FIG. 3 is a plan view of another example medical device; and

FIG. 4 is a plan view of another example medical device.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

Lymphedema is generally characterized by swelling, especially in subcutaneous fatty tissue, as a result of an obstruction of lymphatic vessels and/or lymph nodes. Consequently, lymph and/or lymph proteins may accumulate, leading to edema, chronic inflammation, and fibrosis in the affected region. Lymphedema is often a complication associated with cancer and cancer treatment and can have long term physical and psychosocial consequences for patients. Treatments for lymphedema generally include therapies that may include drainage of the affected area, compression, physical therapy, pharmaceuticals, etc. While some success can be achieved using these and other therapies, an ongoing need exists for alternative treatments.

Disclosed herein are example medical devices and methods for using medical devices that may be used to treat, for example, lymphedema. In general, the devices may be used to actively move fluid including lymphatic fluid within the patent. The devices may be implanted or partially implanted at or adjacent to the lymphatic system (e.g., within a lymphatic vessel) and may include a pump that helps to move the lymphatic fluid. Some additional details of these and other devices are disclosed herein.

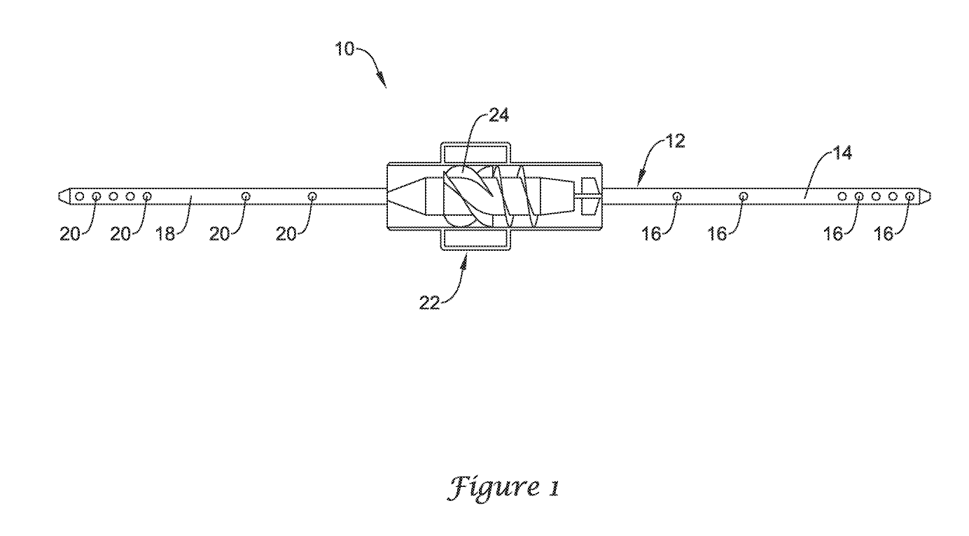

FIG. 1 is a partially cross-sectional side view of an example lymphatic medical device 10. In general, medical device 10 may include an implantable member 12. Implantable member 12 may include a first catheter or catheter portion 14. First catheter portion 14 may have one or more openings 16 formed therein. Implantable member 12 may also include a second catheter or catheter portion 18 having one or more openings 20 formed therein. As shown, openings 16/20 may be longitudinally spaced along catheter portions 14/18. However, other arrangements are contemplated. For example, openings 16/20 may be positioned circumferentially around catheter portions 14/18. In addition, the number and/or arrangement of openings 16/20 need not be the same for both catheter portions 14/18. Numerous other variations are contemplated.

A pump or pump member 22 may be coupled to implantable member 12 and may be generally disposed between first catheter portion 14 and second catheter portion 18. In general, pump 22 may be configured to assist with the movement of fluids through implantable member 12. The form of pump 22 may vary. For example, in at least some embodiments, pump 22 may include an impeller 24. However, other pumps are contemplated including hydraulic pumps, gear or screw pumps, piston type pumps, and the like. These are just examples.

In at least some embodiments, first catheter portion 14 may function as a fluid intake or inlet catheter. Consequently, when implantable member 12 is implanted within a patient (e.g., within a lymphatic vessel) pump 22 may be used to draw fluid (e.g., lymphatic fluid) into implantable member 12 through openings 16 in first catheter portion 14. Second catheter portion 18 may function as a fluid outlet such that pump 22 may transfer lymphatic fluid out from implantable member 12 through openings 20 in second catheter portion 18.

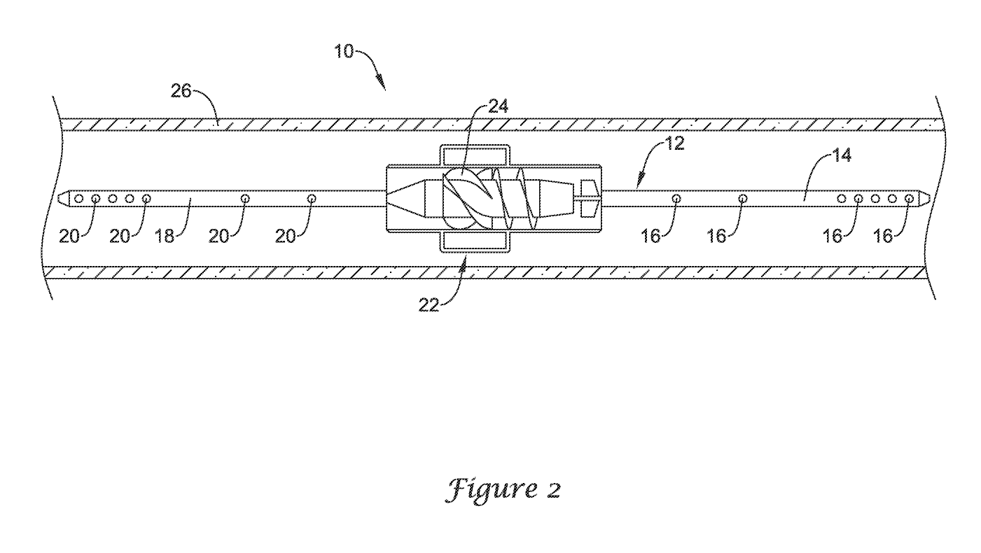

In use, device 10 may be implanted with a body lumen 26 as illustrated in FIG. 2. In at least some embodiments, body lumen 26 may be a lymphatic vessel. However, other locations are contemplated including locations adjacent to a lymphatic vessel, blood vessels, other body lumens, within swollen tissue, and the like. When suitable implanted, device 10 may be used to move lymphatic fluid from one location to another. This may include using pump 22 to draw fluid into implantable member 12 through openings 16 in first catheter portion 14 and out from implantable member 12 through openings 20 in second catheter portion 18.

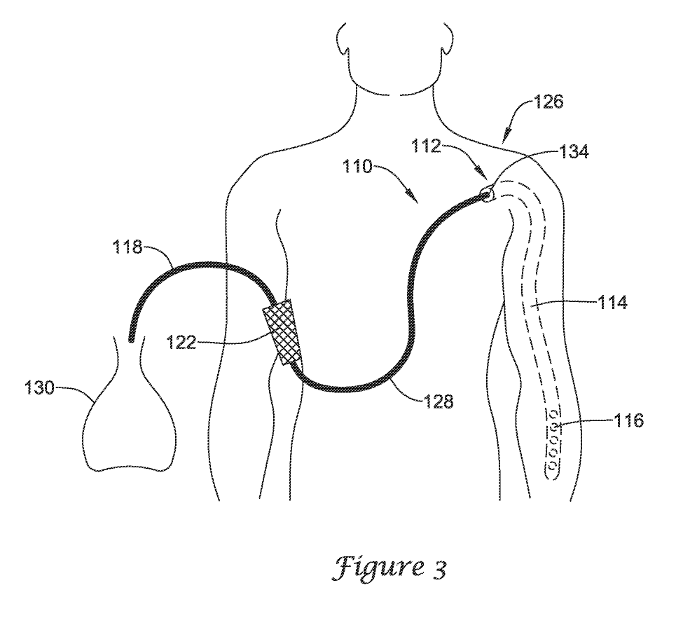

FIG. 3 is a schematic view of another example medical device 110 that may be similar in form and function to other devices disclosed herein. Device 110 may include implantable member 112. Implantable member 112 may include catheter portion 114 having openings 116 formed therein.

Device 110 may also include pump 122, which may be an external pump that is generally positioned along the exterior of the patient 126. Pump 122 may be coupled to an outlet tube 118 that extends to a fluid collection vessel 130. Pump 122 may also be coupled to implantable member 112 via an intermediate tube 128. In at least some embodiments, pump 122 may be detachably connected to implantable member 112. For example, pump 122 may be detachably connected (e.g., via intermediate tube 128) to implantable member 112 at a suitable access port 134.

The use of device 110 may include implanting implantable member 112 within or otherwise adjacent to a lymphatic vessel of a patient 126. This may include disposing access port 134 at a convenient location along, for example, the skin of the patient 126. When suitably implanted, pump 122 may be attached to implantable member 112. This may include attaching tube 128 to access port 134. Activation of pump 122 may draw fluid (e.g., lymphatic fluid) into implantable member 112 through openings 116 of catheter portion 114. The fluid may be transported to fluid collection vessel 130.

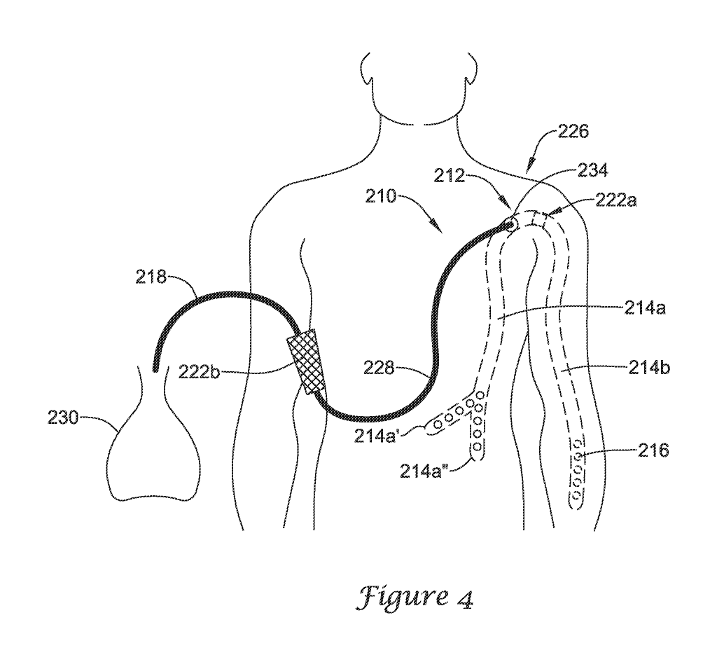

FIG. 4 is a schematic view of another example medical device 210 that may be similar in form and function to other devices disclosed herein. Device 210 may include implantable member 212. Implantable member 212 may have one or more branches formed therein. For example, implantable member 212 may include catheter portion 214a and catheter portion 214b. In some of these and in other embodiments, one or both of catheter portions 214a/214b may be further branched. For example, catheter portion 214a may include catheter branches 214a′/214a″. The number and/or configuration of the branches can vary. Any of catheter portions/branches 214a/214a′/214a″/214b may have one or more having openings 216 formed therein.

In at least some embodiments, implantable member 212 may include pump 222a. Pump 222a may be similar in form and function to other pumps disclosed herein. For example, pump 222a may be an implantable pump similar to pump 22. In some embodiments, device 210 may lack pump 222a. Device 210 may also include external pump 222b, which may be generally positioned along the exterior of the patient 226. Pump 222b may be coupled to an outlet tube 218 that extends to a fluid collection vessel 230. Pump 222b may also be coupled to implantable member 212 via an intermediate tube 228. In at least some embodiments, pump 222b may be detachably connected to implantable member 212. For example, pump 222b may be detachably connected (e.g., via intermediate tube 228) to implantable member 212 at access port 234.

The use of device 210 may include implanting implantable member 212 within or otherwise adjacent to a lymphatic vessel of a patient 226. This may include disposing access port 234 at a convenient location along, for example, the skin of the patient 226. At this point, pump 222a may be utilized to transfer fluid from one location to another along implantable member 212. For example, pump 222a may be used to transfer fluid from a position adjacent to catheter portion 214a to a position adjacent to catheter portion 214b, or vice versa. Pump 222b may be used in conjunction with pump 222a or pump 222b may be used instead of pump 222a (e.g., in embodiments where device 210 lacks pump 222a). For example, pump 222b may be attached to implantable member 212. This may include attaching tube 228 to access port 234. Activation of pump 222a may draw fluid (e.g., lymphatic fluid) into implantable member 212 through openings 216 of catheter portion 214a/214b. The fluid may be transported to fluid collection vessel 230.

The materials that can be used for the various components of device 10 (and/or other devices disclosed herein) and the various tubular members disclosed herein may include those commonly associated with medical devices. For simplicity purposes, the following discussion makes reference to implantable member 12 and other components of device 10. However, this is not intended to limit the devices and methods described herein, as the discussion may be applied to other similar tubular members and/or components of tubular members or devices disclosed herein.

Implantable member 12 and/or other components of device 10 may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also can be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and

DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, portions or all of device 10 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of device 10 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of device 10 to achieve the same result.

In some embodiments, a degree of Magnetic Resonance Imaging (MRI) compatibility is imparted into device 10. For example, implantable member 12, or portions thereof, may be made of a material that does not substantially distort the image and create substantial artifacts (i.e., gaps in the image). Certain ferromagnetic materials, for example, may not be suitable because they may create artifacts in an MRI image. Implantable member 12, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A medical device for moving lymphatic fluid, comprising:
an implantable body having an inlet end region and an outlet end region; and
a pump member positioned between the inlet end region and the outlet end region, the pump member being configured to draw lymphatic fluid into the implantable body through the inlet end region and transfer lymphatic fluid out from the implantable body through the outlet end region.

2. The medical device of claim 1, wherein the implantable body includes a first catheter portion and a second catheter portion, the inlet end region being positioned along the first catheter portion and the outlet end region being positioned along the second catheter portion.

3. The medical device of claim 2, wherein the first catheter portion, the second catheter portion, or both include a plurality of catheter branches.

4. The medical device of claim 1, wherein the inlet end region, the outlet end region, and the pump member are configured to be at least partially implanted within a lymphatic vessel of a patient.

5. The medical device of claim 1, wherein the pump member includes an external pump that is configured to be positioned along an outer surface of a patient.

6. The medical device of claim 5, wherein the external pump is detachably connected to the implantable body.

7. The medical device of claim 5, wherein the implantable body includes a catheter portion having a plurality of branches formed therein, the inlet end region being positioned along the catheter portion.

8. The medical device of claim 5, further comprising a fluid collection member coupled to the pump member.

9. The medical device of claim 8, wherein the outlet end region is positioned adjacent to the fluid collection member.

10. The medical device of claim 1, wherein the pump member includes an impeller.

11. A lymphedema medical device, comprising:
an implantable tubular member having one or more fluid openings formed therein;
wherein the implantable tubular member is configured to be implanted within a lymphatic vessel region of a patient;
a pump coupled to the implantable tubular member, the pump being configured to draw lymphatic fluid into the implantable tubular member through the one or more fluid openings; and
an outlet tube coupled to the pump.

12. The lymphedema medical device of claim 11, wherein the pump includes an implantable pump configured to be implanted within the patient.

13. The lymphedema medical device of claim 12, wherein the outlet tube includes a second implantable tubular member that is configured to be implanted within a second lymphatic vessel region of the patient.

14. The lymphedema medical device of claim 11, wherein the pump includes an external pump.

15. The lymphedema medical device of claim 14, wherein the outlet tube extends to a fluid collection vessel.

16. The lymphedema medical device of claim 11, wherein the implantable tubular member has a plurality of branches formed therein.

17. The lymphedema medical device of claim 11, wherein the pump includes an implantable pump and an external pump.

18. A method for moving lymphatic fluid, the method comprising:
providing a lymphedema medical device, the lymphedema medical device comprising:
an implantable tubular member having one or more fluid openings formed therein,
a pump coupled to the implantable tubular member, the pump being configured to draw lymphatic fluid into the implantable tubular member through the one or more fluid openings, and
an outlet tube coupled to the pump;
implanting the implantable tubular member within a lymphatic vessel region of a patient; and
activating the pump, wherein activating the pump draws lymphatic fluid into the implantable tubular member through the one of more fluid openings and to the outlet tube.

19. The method of claim 18, wherein the outlet tube is implanted within a second lymphatic vessel region of the patient, and wherein activating the pump transfers lymphatic fluid to the second lymphatic vessel region.

20. The method of claim 18, wherein the outlet tube is coupled to a fluid collection vessel, and wherein activating the pump transfers lymphatic fluid to the fluid collection vessel.
